(19) United States
(12) Patent Application Publication
(10) Pub. No.: US 2005/0026910 A1
(43) Pub. Date: Feb. 3, 2005

(54) Method of treating viral diseases

(75) Inventors: William Roush, Ann Arbor, MI (US); Nan Zheng, Ann Arbor, MI (US); Vassil St. Georgiev, Gaithersburg, MD (US)

Correspondence Address: MEDLEN & CARROLL, LLP, Suite 350, 101 Howard Street, San Francisco, CA, 94105, US

(73) Assignee: The Regents of the University of Michigan, Ann Arbor, MI (US)

(21) Appl. No.: 10/878,339

(22) Filed: Jun. 28, 2004

Related U.S. Application Data
(60) Provisional application No. 60/490,214, filed on Jul. 25, 2003.

(51) Int. Cl. A61K 31/551

(52) U.S. Cl. 514/219; 514/220

(57) ABSTRACT

The present invention relates to novel chemical compounds, methods for their discovery, and therapeutic use. In particular, the present invention provides benzimidazole derivatives and methods of using benzimidazole derivatives as therapeutic agents to treat a number of conditions associated with retroviral infection.

This invention was supported in part with NIH grant AI 35709. The United States government may have rights in this invention.

This application claims priority to U.S. Provisional Patent Ser. No. 60/490,214, filed Jul. 25, 2003.

### FIELD OF THE INVENTION

The present invention relates to novel chemical compounds, methods for their discovery, and their therapeutic use. In particular, the present invention provides benzimidazole derivatives and related compounds and methods of using benzimidazole derivatives and related compounds as therapeutic agents to treat a number of conditions associated with viral infection, and the like.

### BACKGROUND OF THE INVENTION

AIDS (acquired immunodeficiency syndrome) is one of the most fatal diseases facing society at the present time. Since it was first diagnosed 20 years ago, more than 22 million people have died from AIDS and another 36 million are living with HIV (human immunodeficiency virus), the causative agent of AIDS.

HIV is an enveloped single-stranded RNA virus and has been identified as a retrovirus of the Lentiviridae family. Two genetically distinct subtypes, HIV-1 and HIV-2, have been found. HIV-1 has been identified as the prevalent cause of AIDS. An anti-HIV agent can exert its activities by interfering at any of these stages in the life cycle of the HIV virus.

Drugs that have been approved for clinical use in the treatment of HIV-1 belong to one of two categories: 1) nucleoside or non-nucleoside reverse transcriptase inhibitors, and 2) protease inhibitors. However, there are two significant problems with current anti-HIV chemotherapy. First, none of the current therapy is potent enough to completely eradicate the virus from latently and chronically infected cells. Secondly, the emergence of drug resistance significantly limits the clinical utility of inhibitors targeted to specific HIV enzymes. Viral resistance is a direct consequence of the variation in HIV genome, which is largely due to the inherent high error rate of reverse transcriptase and the high replication levels of HIV virus in vivo.

What is needed are methods of HIV-1 treatment that are capable of targeting the virus within latently and chronically infected cells. What also is needed is a method that reduces the likelihood of viral drug resistance.

### SUMMARY

The present invention relates to novel chemical compounds, methods for their discovery, and therapeutic use. In particular, the present invention provides benzimidazole derivatives and methods of using benzimidazole derivatives. In addition, the present invention also provides uses of these novel compounds in order to elicit particular biological responses (e.g., inhibiting retroviral life cycle) as therapeutic agents to treat a number of conditions associated with retroviral infection. Such compounds and uses are described throughout the present application and represent a diverse collection of compositions and applications.

Certain preferred compositions and uses are described below. The present invention is not limited to these particular compositions and uses.

The present invention provides a number of useful compositions as described throughout the present application. Certain preferred embodiments of the present involve compositions include a composition comprising the following formula: 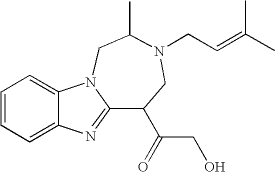
including its enantiomer and racemic mixtures.

Other preferred embodiments of the present involve compositions include a composition comprising the following formula: 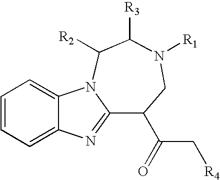
including its enantiomer and racemic mixtures; wherein R1 comprises a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxyl group, an orthophosphate group, a diphospate group, or a triphosphate group.

Other preferred embodiments of the present involve compositions include a composition comprising the following formula: 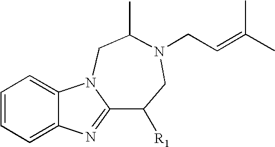
including its enantiomer and racemic mixtures; wherein R1 is a hydroxy-methyl-ketone, an orthophosphate-methyl-ketone, a diphosphate-methyl-ketone, or a triphosphate-methyl-ketone.

Other preferred embodiments of the present involve compositions include a composition comprising the following formula: 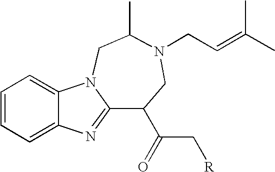
including its enantiomers and racemic mixtures; wherein R is a hydroxyl group, an orthophosphate group, a diphospate group, or a triphosphate group.

Other preferred embodiments of the present involve compositions include a composition comprising the following formula: 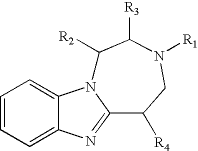
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone.

The present invention also provides methods and compositions useful in inhibiting a retroviral life cycle. In preferred embodiments, the present invention provides target cells and a composition, and the target cells are exposed to the composition. In further embodiments, proliferation and apoptosis are measured within the target cells following exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition comprises the following formula: 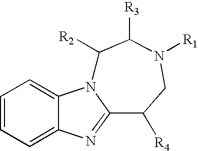
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 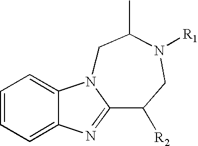
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 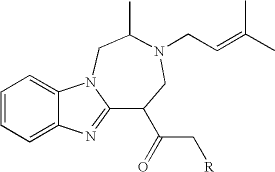
including its enantiomers and racemic mixtures; wherein R comprises a hydroxyl group, an orthophosphate group, a diphospate group, or a triphosphate group. In still other preferred embodiments, the composition comprises the following formula: 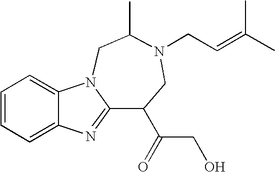
including its enantiomer and racemic mixtures.

The present invention also provides methods and compositions useful in inhibiting a pre-translational retroviral life cycle. In preferred embodiments, the present invention provides target cells comprising reverse transcriptase and a composition, and the target cells are exposed to the composition under conditions such that a binding occurs between the composition and the reverse transcriptase. In further embodiments, proliferation and apoptosis are measured within the target cells following exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition comprises the following formula: 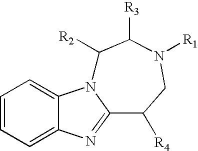
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 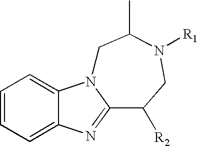
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 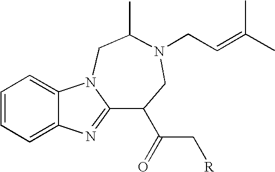
including its enantiomers and racemic mixtures; wherein R comprises a hydroxyl group, an orthophosphate group, a diphospate group, or a triphosphate group. In still other preferred embodiments, the composition comprises the following formula: 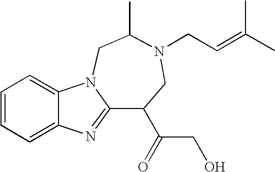
including its enantiomer and racemic mixtures.

The present invention also provides methods and compositions useful in inhibiting retroviral reverse transcriptase. In preferred embodiments, the present invention provides target cells comprising reverse transcriptase and a composition, and the target cells are exposed to the composition under conditions such that a binding occurs between the composition and the reverse transcriptase. In further embodiments, the activity levels of reverse transcriptase within the target cells are measured after exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition comprises the following formula: 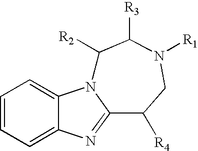
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 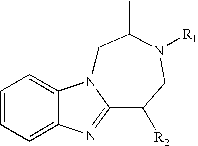
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 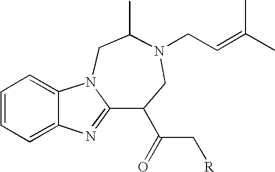
including its enantiomers and racemic mixtures; wherein R comprises a hydroxyl group, an orthophosphate group, a diphospate group, or a triphosphate group. In still other preferred embodiments, the composition comprises the following formula: 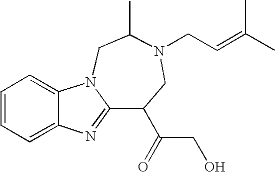
including its enantiomer and racemic mixtures.

The present invention also provides methods and compositions useful in inhibiting retroviral DNA synthesis. In preferred embodiments, the present invention provides target cells comprising reverse transcriptase and a composition, and the target cells are exposed to the composition under conditions such that a binding occurs between the composition and the reverse transcriptase. In further embodiments, DNA synthesis is measured within the target cells are measured after exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition comprises the following formula: 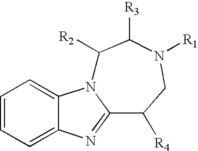
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 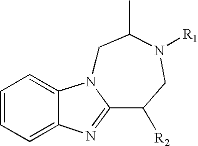
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 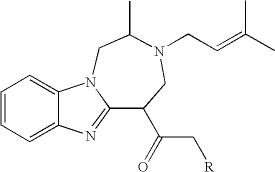
including its enantiomers and racemic mixtures; wherein R comprises a hydroxyl group, an orthophosphate group, a diphospate group, or a triphosphate group. In still other preferred embodiments, the composition comprises the following formula: 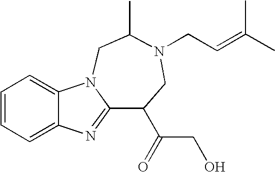
including its enantiomer and racemic mixtures.

The present invention also provides methods and compositions useful in inhibiting a post-translational retroviral life cycle. In preferred embodiments, the present invention provides target cells comprising TAT protein and a composition, and the target cells are exposed to the composition under conditions such that a binding occurs between the composition and the TAT protein. In further embodiments, proliferation and apoptosis within the target cells are measured after exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition inhibits TAT protein from binding with retroviral RNA. In other preferred embodiments, the composition comprises the following formula: 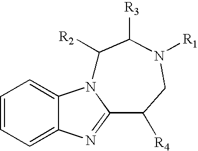
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 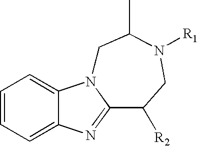
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 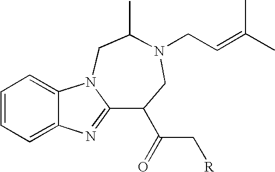
including its enantiomers and racemic mixtures; wherein R comprises a hydroxyl group, an orthophosphate group, a diphospate group, or a triphosphate group. In still other preferred embodiments, the composition comprises the following formula: 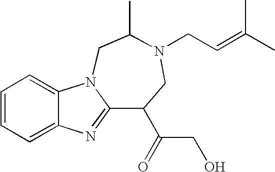
including its enantiomer and racemic mixtures.

The present invention also provides methods and compositions useful in inhibiting retroviral TAT protein. In preferred embodiments, the present invention provides target cells comprising TAT protein and a composition, and the target cells are exposed to the composition under conditions such that a binding occurs between the composition and the TAT protein. In further embodiments, TAT levels are measured within the target cells are measured after exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition inhibits TAT protein from binding with retroviral RNA. In other preferred embodiments, the composition comprises the following formula: 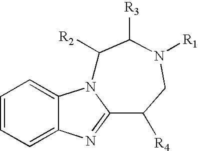
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 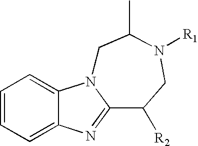
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 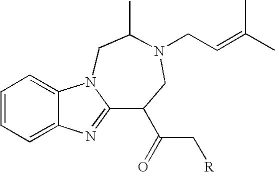
including its enantiomers and racemic mixtures; wherein R comprises a hydroxyl group, an orthophosphate group, a diphospate group, or a triphosphate group. In still other preferred embodiments, the composition comprises the following formula: 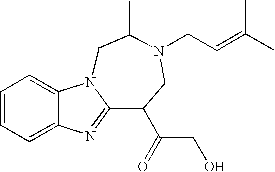
including its enantiomer and racemic mixtures.

The present invention also provides methods and compositions useful in inhibiting retroviral RNA synthesis. In preferred embodiments, the present invention provides target cells comprising TAT protein and a composition, and the target cells are exposed to the composition under conditions such that a binding occurs between the composition and the TAT protein. In further embodiments, RNA synthesis is measured within the target cells are measured after exposure to the composition.

In preferred embodiments, the target cells comprise HIV-1, and are in vitro, in vivo, or ex vivo cells.

In preferred embodiments, the composition functions through inhibiting viral DNA Polymerase and binding viral reverse transcriptase. In particularly preferred embodiments, compositions of the present invention bind the palm domain of the p66 subunit of reverse transcriptase. In other preferred embodiments, the composition inhibits TAT protein from binding with retroviral RNA. In other preferred embodiments, the composition comprises the following formula: 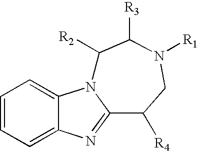
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the composition comprises the following formula: 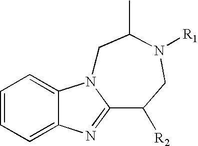
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 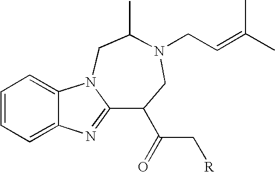
including its enantiomers and racemic mixtures; wherein R comprises a hydroxyl group, an orthophosphate group, a diphospate group, or a triphosphate group. In still other preferred embodiments, the composition comprises the following formula: 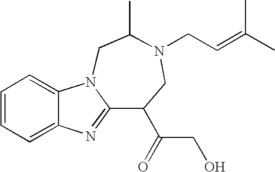
including its enantiomer and racemic mixtures.

The present invention also provides methods and compositions useful in treating a retroviral disorder. In preferred embodiments, the present invention provides a subject suffering from a retroviral disorder and a compound, and the compound is administered to the subject. In further embodiments, the retroviral disorder is AIDS. In other preferred embodiments, the retroviral disorder is T-Cell Lymphoma.

In some embodiments, an antiretroviral agent is provided. In preferred embodiments, the antiretroviral agent is administered along with the compound to the subject. In further embodiments, the antiviral agent is in said antiretroviral agent is selected from the group consisting of Agenerase (amprenavir), Combivir, Crixivan (indinavir), Epivir (3tc/lamivudine), Emtriva (emtricitabine (FTC)), Fortovase (saquinavir), Kaletra (lopinavir), Norvir (ritonavir), Rescriptor (delavirdine), Retrovir, AZT (zidovudine), Reyataz (atazanavir), Sustiva (efavirenz), Trizivir, Videx, Videx EC (ddl/didanosine), Viracept (nelfinavir), Viramune (nevirapine), Viread (tenofovir disoproxil fumarate), Zerit (d4t/stavudine), and Ziagen (abacavir).

In preferred embodiments, the compound functions through inhibiting viral DNA Polymerase and binding viral reverse transcriptase. In particularly preferred embodiments, compounds of the present invention bind the palm domain of the p66 subunit of reverse transcriptase. In other preferred embodiments, the compound inhibits TAT protein from binding with retroviral RNA. In other preferred embodiments, the compound comprises the following formula: 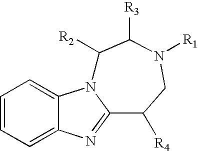
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone. In yet other preferred embodiments, the compound comprises the following formula: 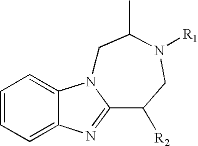
including its enantiomer and racemic mixtures; wherein R1 comprises a branched alkane, a branched alkene, or a branched alkyne; wherein R2 comprises a hydroxy-methyl-ketone, an orthophosphate-methyl-ketone, a diphosphate-methyl-ketone, or a triphosphate-methyl-ketone. In still other embodiments, the composition comprises the following formula: 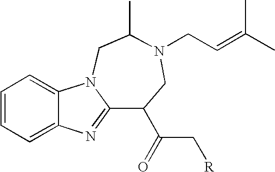
including its enantiomers and racemic mixtures; wherein R comprises a hydroxyl group, an orthophosphate group, a diphospate group, or a triphosphate group. In still other preferred embodiments, the compound comprises the following formula: 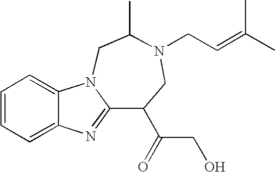
including its enantiomer and racemic mixtures.

In other preferred embodiments, the present invention provides a pharmaceutical composition. In such embodiments, the pharmaceutical composition comprises a nucleoside reverse transcriptase inhibitor. In other preferred embodiments, the pharmaceutical composition further comprises a therapeutic agent. In still further preferred embodiments, the therapeutic agent is selected from the group consisting of Agenerase (amprenavir), Combivir, Crixivan (indinavir), Epivir (3tc/lamivudine), Emtriva (emtricitabine (FTC)), Fortovase (saquinavir), Kaletra (fopinavir), Norvir (ritonavir), Rescriptor (delavirdine), Retrovir, AZT (zidovudine), Reyataz (atazanavir), Sustiva (efavirenz), Trizivir, Videx, Videx EC (ddl/didanosine), Viracept (nelfinavir), Viramune (nevirapine), Viread (tenofovir disoproxil fumarate), Zerit (d4t/stavudine), and Ziagen (abacavir). In preferred embodiments, the nucleoside reverse transcriptase inhibitor comprises the following structure: 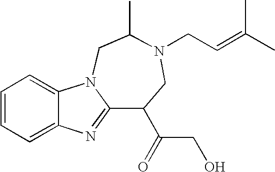
including its enantiomers and racemic mixtures. In other preferred embodiments, the nucleoside reverse transcriptase inhibitor comprises the following structure: 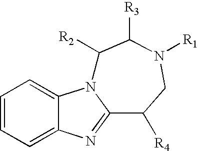
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone.

In preferred embodiments, the pharmaceutical composition provided by the present invention comprises a non-nucleoside reverse transcriptase inhibitor. In other preferred embodiments, the pharmaceutical composition further comprises a therapeutic agent. In still further preferred embodiments, the therapeutic agent is selected from the group consisting of Agenerase (amprenavir), Combivir, Crixivan (indinavir), Epivir (3tc/lamivudine), Emtriva (emtricitabine (FTC)), Fortovase (saquinavir), Kaletra (lopinavir), Norvir (ritonavir), Rescriptor (delavirdine), Retrovir, AZT (zidovudine), Reyataz (atazanavir), Sustiva (efavirenz), Trizivir, Videx, Videx EC (ddl/didanosine), Viracept (nelfinavir), Viramune (nevirapine), Viread (tenofovir disoproxil fumarate), Zerit (d4t/stavudine), and Ziagen (abacavir). In preferred embodiments, the non-nucleoside reverse transcriptase inhibitor comprises the following structure: 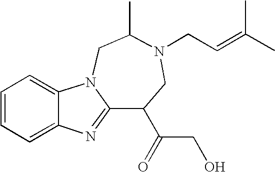
including its enantiomers and racemic mixtures. In other preferred embodiments, the non-nucleoside reverse transcriptase inhibitor comprises the following structure: 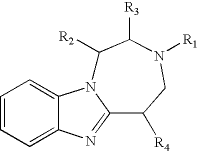
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone.

The present invention also provides methods and compositions useful in identifying pharmaceutical agents useful for treating retroviral disorders. In preferred embodiments, the present invention provides target cells comprising reverse transcriptase and TAT protein, and a candidate agent, exposing the target cells to the candidate agents, measuring RNA synthesis of the retroviral cells, the activity of said TAT protein of the retroviral cells, the activity of the reverse transcriptase, and the proliferation and apoptosis of the retroviral cells, and selecting candidate pharmaceutical agents that inhibit RNA synthesis, inhibit the activity of the TAT protein, inhibit the activity of the reverse transcriptase, inhibit proliferation of the retroviral cells, and induce apoptosis of the retroviral cells. In preferred embodiments, the target cells are HIV-1 cells.

Definitions

To facilitate an understanding of the present invention, a number of terms and phrases are defined below.

As used herein, the term “benzimidazole” refers to an imidazole ring fused to a phenyl ring. In some aspects, the two nitrogen atoms within the imidazole ring are in 1 and 3 positions, as shown in the general structure below.

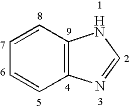

The benzimidazole can be substituted. Generally, the benzimidazole is further substituted either on the six-membered phenyl ring or on the imidazole ring or on both rings by a variety of substituents. These substituents are described more fully herein.

As used herein, the term “aliphatic” or “aliphatic chain” refers to a class of organic compounds where carbon and hydrogen molecules are arranged in straight or branched chains. The chain may include saturated (e.g., alkanes) or unsatured (e.g., alkenes and alkynes) elements. Examples include, but are not limited to, ethane, ethene, ethyne, octane, 2-octene, 2-octyne, pentadecane, hexadecane, and eicosane.

As used herein, the term “substituted aliphatic” or “substituted aliphatic chain” refers to an aliphatic chain where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of no more than three fused rings at least one of which is aromatic, and where at least one of the hydrogen atoms on a ring carbon has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to a cycloalkane or a fused ring system consisting of at least one fused cycloaliphatic ring. Examples of such include, but are not limited to, decalin and the like.

As used herein, the term “substituted cycloaliphatic” refers to a cycloalkane or a fused ring system consisting of at least one fused ring, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl and the like.

As used herein, the term “heterocyclic” refers to a cycloalkane and/or an aryl ring system and/or a fused ring system consisting of at least one fused ring, where at least one of the ring carbon atoms is replaced by oxygen, nitrogen or sulfur. Examples of such include, but are not limited to, morpholino and the like.

As used herein, the term “substituted heterocyclic” refers to a cycloalkane and/or an aryl ring system and/or a fused ring system consisting of at least one fused ring, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 3-chloropyranyl.

As used herein, the term “nitro” or “nitro subgroup” refers to an N0₂ subgroup. Examples of compounds containing nitro subgroups include, but are not limited to, nitrobenzene.

As used herein, the term “linker” refers to a chain containing at least two contiguous atoms connecting two different structural moieties where such atoms are, for example, carbon, nitrogen, oxygen, or sulfur. Ethylene glycol is one non-limiting example.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

As used herein, the term “acylamino” is an amino group that has been acylated. Examples of such include, but are not limited to, acetamide and the like.

The term “derivative” of a compound, as used herein, refers to a chemically modified compound wherein the chemical modification takes place either at a functional group of the compound or on the benzimidazole core structure.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of benzimidazole compound(s), and optionally one or more other agents) for a condition characterized by viral infection.

The term “diagnosed,” as used herein, refers to the to recognition of a disease by its signs and symptoms (e.g., resistance to conventional therapies), or genetic analysis, pathological analysis, histological analysis, and the like.

As used herein, the term “virus” refers to obligate intracellular parasites of living but noncellular nature. Examples include, but are not limited to, HIV-1, HTLV-1, Human Herpes Virus 6, and Hepatitis A Virus.

As used herein, the term “retrovirus” refers to any virus in the family Retroviridae that has RNA has its nucleic acid and uses the enzyme reverse transcriptase to copy its genome into the DNA of the host cell chromosomes.

As used herein, the term “viral disease” or “viral infection” or “viral disorder” or “viral condition” refer to any disease, infection, condition, or disorder caused or exacerbated by a virus. Examples include, but are not limited to, human immunodeficiency virus—1 (HIV-1), acquired immunodeficiency syndrome (AIDS), herpes simplex virus (HSV), varicella zoster virus (VZV), respiratory syncytial virus (RSV) and cytomegalovirus (CMV).

As used herein, the term “retroviral disease” or “retroviral infection” or “retroviral disorder” or “retroviral condition” refer to any disease, infection, condition, or disorder caused or exacerbated by a retrovirus. Examples include, but are not limited, AIDS, T-cell leukemia, and T-cell lymphoma.

As used herein, the terms “antiviral agent,” or “conventional antiviral agent” refer to any chemotherapeutic compounds used in the treatment of viral disorders. Examples include, but are not limited to, Agenerase (amprenavir), Combivir, Crixivan (indinavir), Epivir (3tc/lamivudine), Emtriva (emtricitabine (FTC)), Fortovase (saquinavir), Fuzeon (enfuvirtide), Hivid (ddc/zalcitabine), Hydrea (hydroxyurea), Invirase (saquinavir), Kaletra (lopinavir), Norvir (ritonavir), Rescriptor (delavirdine), Retrovir, AZT (zidovudine), Reyataz (atazanavir), Sustiva (efavirenz), Trizivir, Videx, Videx EC (ddl/didanosine), Viracept (nelfinavir), Viramune (nevirapine), Viread (tenofovir disoproxil fumarate), Zerit (d4t/stavudine), and Ziagen (abacavir).

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “viral cells” or “cells infected with virus” or “viral infected cells” refers to any type of cell infected with any type of virus. Examples include, but are not limited to human monocytes infected with HIV-1, and human adult T-cells infected with HTLV-1.

In preferred embodiments, the “target cells” of the compositions and methods of the present invention include, refer to, but are not limited to, viral cells, viral infected cells, or cells infected with a virus. In some embodiments, target cells are continuously cultured cells or uncultered cells obtained from patient biopsies.

In one specific embodiment, the target cells exhibit pathological growth or proliferation. As used herein, the term “pathologically proliferating or growing cells” refers to a localized population of proliferating cells in an animal that is not governed by the usual limitations of normal growth.

As used herein, the term “un-activated target cell” refers to a cell that is either in the Gₒ phase or one in which a stimulus has not been applied.

As used herein, the term “activated target lymphoid cell” refers to a lymphoid cell that has been primed with an appropriate stimulus to cause a signal transduction cascade, or alternatively, a lymphoid cell that is not in Gₒ phase. Activated lymphoid cells may proliferate, undergo activation induced cell death, or produce one or more of cytotoxins, cytokines, and other related membrane-associated proteins characteristic of the cell type (e.g., CD8⁺ or CD4⁺). They are also capable of recognizing and binding any target cell that displays a particular antigen on its surface, and subsequently releasing its effector molecules.

Examples of a T cell ligand include, but are not limited to, a peptide that binds to an MHC molecule, a peptide MHC complex, or an antibody that recognizes components of the T cell receptor.

Examples of a B cell ligand include, but are not limited to, a molecule or antibody that binds to or recognizes components of the B cell receptor.

Examples of agents or conditions that enhance cell stress include heat, radiation, oxidative stress, or growth factor withdrawal and the like. Examples of growth factors include, but are not limited to serum, IL-2, platelet derived growth factor (“PDGF”), and the like.

As used herein, the term “effective amount” refers to the amount of a compound (e.g., benzimidazole) sufficient to effect beneficial or desired results. An effective amount can be administered in one or more administrations, applications or dosages and is not limited to or intended to be limited to a particular formulation or administration route.

As used herein, the term “dysregulation of the process of cell death” refers to any aberration in the ability of (e.g., predisposition) a cell to undergo cell death via either necrosis or apoptosis. Dysregulation of cell death is associated with or induced by a variety of conditions, including for example, autoimmune disorders (e.g., systemic lupus erythematosus, rheumatoid arthritis, graft-versus-host disease, myasthenia gravis, Sjögren's syndrome, etc.), chronic inflammatory conditions (e.g., psoriasis, asthma and Crohn's disease), hyperproliferative disorders (e.g., tumors, B cell lymphomas, T cell lymphomas, etc.), viral infections (e.g., herpes, papilloma, HIV), and other conditions such as osteoarthritis and atherosclerosis.

It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

A “hyperproliferative disorder,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A metastatic cell or tissue means that the cell can invade and destroy neighboring body structures. Hyperplasia is a form of cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include rheumatoid arthritis, Sjögren's syndrome, graft versus host disease, myasthenia gravis, systemic lupus erythematosus (“SLE”), and the like.

As used herein, the term “chronic inflammatory condition” refers to a condition wherein the organism's immune cells are activated. Such a condition is characterized by a persistent inflammatory response with pathologic sequelae. This state is characterized by infiltration of mononuclear cells, proliferation of fibroblasts and small blood vessels, increased connective tissue, and tissue destruction. Examples of chronic inflammatory diseases include, but are not limited to, Crohn's disease, psoriasis, chronic obstructive pulmonary disease, inflammatory bowel disease, multiple sclerosis, and asthma. Autoimmune diseases such as rheumatoid arthritis and systemic lupus erythematosus can also result in a chronic inflammatory state.

As used herein, the term “co-administration” refers to the administration of at least two agent(s) (e.g., benzimidazoles) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, asp artate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “solid phase supports” or “solid supports,” are used in their broadest sense to refer to a number of supports that are available and known to those of ordinary skill in the art. Solid phase supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid phase support may be selected on the basis of desired end use and suitability for various protocols. For example, for peptide synthesis, solid phase supports may refer to resins such as polystyrene (e.g., PAM-resin obtained from Bachem, Inc., Peninsula Laboratories, etc.), POLYHIPE resin (obtained from Aminotech, Canada), polyamide resin (obtained from Peninsula Laboratories), polystyrene resin grafted with polyethylene glycol (TENTAGEL, Rapp Polymere, Tubingen, Germany) or polydimethylacrylamide resin (obtained from Milligen/Biosearch, California).

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms which are gram negative or gram positive. “Gram negative” and “gram positive” refer to staining patterns with the Gram-staining process which is well known in the art. (See e.g., Finegold and Martin, Diagnostic Microbiology, 6th Ed., CV Mosby St. Louis, pp. 13-15 [1982]). “Gram positive bacteria” are bacteria which retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

The term “sample” as used herein is used in its broadest sense. A sample suspected of containing retroviruses or retroviral components may comprise a cell, tissue, or fluids, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin [KLH]). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc.,pp. 77-96 [1985]).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

The term “epitope” as used herein refers to that portion of an antigen that makes contact with a particular immunoglobulin. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “modulate” refers to the activity of a compound (e.g., benzimidazole compound) to affect (e.g., to promote or retard) an aspect of cellular function, including, but not limited to, cell growth, proliferation, apoptosis, and the like.

As used herein, the term “competes for binding” is used in reference to a first molecule (e.g., a first benzimidazole derivative) with an activity that binds to the same substrate (e.g., viral reverse transcriptase) as does a second molecule (e.g., a second benzimidazole derivative or other molecule that inhibits reverse transcriptase, etc.). The efficiency (e.g., kinetics or thermodynamics) of binding by the first molecule may be the same as, or greater than, or less than, the efficiency of the substrate binding to the second molecule. For example, the equilibrium binding constant (K_{D}) for binding to the substrate may be different for the two molecules.

As used herein, the term “instructions for administering said compound to a subject,” and grammatical equivalents thereof, includes instructions for using the compositions contained in a kit for the treatment of conditions characterized by viral infection (e.g., providing dosing, route of administration, decision trees for treating physicians for correlating patient-specific characteristics with therapeutic courses of action).

The term “test compound” refers to any chemical entity, pharmaceutical, drug, and the like, that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention.

As used herein, the term “third party” refers to any entity engaged in selling, warehousing, distributing, or offering for sale a test compound contemplated for administered with a compound for treating conditions characterized by the dysregulation of apoptotic processes.

### DETAILED DESCRIPTION OF THE INVENTION

The present invention provides novel chemical compounds, methods for their discovery, and their therapeutic use. In particular, the present invention provides benzimidazole derivatives and related compounds and methods of using benzimidazole derivatives and related compounds as therapeutic agents to treat a number of conditions associated with viral infection, and the like.

Exemplary compositions and methods of the present invention are described in more detail in the following sections: I. Modulators of Antiviral Activity; II. Exemplary Compounds; III. Pharmaceutical compositions, formulations, and exemplary administration routes and dosing considerations; IV. Drug screens; and V. Therapeutic Applications.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is herein incorporated by reference in its entirety.

I. Modulators of Antiviral Activity

In preferred embodiments, the present invention provides antiviral activity through the exposure of viruses to compounds. In particularly preferred embodiments, the present invention provides antiviral activity through the exposure of retroviruses (e.g., HIV-1) to compounds. The effect of the compounds can be measured by detecting any number of cellular changes. Viral cell replication may be assayed as described herein and in the art. Viral cell death may be assayed as described herein and in the art. In preferred embodiments, viral cell lines are maintained under appropriate cell culturing conditions (e.g., gas (CO₂), temperature and media) for an appropriate period of time to attain exponential proliferation without density dependent constraints. Viral cell number and or viability are measured using standard techniques (e.g., trypan blue exclusion/hemo-cytometry, or MTT dye conversion assay). Alternatively, the viral cell may be analyzed for the expression of genes or gene products associated with aberrations in apoptosis or necrosis or replication.

In preferred embodiments, the present invention provides methods and compositions for inhibiting pre-translational stages of the retroviral life cycle. In other preferred embodiments, the present invention provides methods and compositions for inhibiting post-translational stages of the retroviral life cycle.

In some embodiments, the present invention provides methods and compositions for inhibiting the pre-translational stages of the retroviral (e.g., HIV-1, HIV-2) life cycle through targeting reverse transcriptase (RT). RT is essential for retroviral replication and is not required for normal cell replication. Two functionally distinct classes of retroviral RT inhibitors include nucleoside reverse transcriptase inhibitors (NRTI) and non-nucleoside reverse transcriptase inhibitors (NNRTI).

In some embodiments, the present invention provides compositions that function as an NRTI. NRTIs are nucleoside analogs and inhibit RT by binding a nucleoside substrate site that is located in the palm subdomain of the RT p66 subunit. In preferred embodiments, the compounds of the present invention targets and binds an RT p66 subunit palm subdomain. NRTIs may inhibit pre-translational replication by competing with the natural substrate. This competition may result in the NRTI acting as a chain terminator in the synthesis of DNA considering that it lacks the 3′-hydroxy group necessary for the formation of phosphodiester linkages. In preferred embodiments, the present invention provides methods and compositions for inhibiting the synthesis of viral DNA synthesis. NRTIs also inhibit other enzymes involved in DNA synthesis such as mammalian DNA polymerases α, β, δ, and γ resulting in cytotoxicity. In some embodiments, the compounds of the present invention function as an inhibitor of DNA synthesis. In some embodiments, the present invention provides methods and compositions for inhibiting mammalian DNA Polymerase (e.g., DNA polymerases α, β, δ, and γ).

In some embodiments, the compounds of the present invention functions as an NNRTI. NNRTIs inhibit RT by binding a common allosteric site that is distinct from the nucleoside substrate site. NNRTIs are non-competitive inhibitors and are highly specific for HIV (e.g., HIV-1, HIV-2). The allosteric site is highly hydrophobic, elastic and is able to adopt a shape complementary to structurally diverse inhibitors. NNRTIs may inhibit RT by locking the polymerase active site in an inactive conformation. In some embodiments, the present invention provides methods and compositions for inhibiting RT through locking the RT polymerase active site in an inactive conformation.

In some embodiments, the present invention provides methods and compositions for inhibiting the post-translational stages of a retroviral (e.g., HIV-1, HTLV-1) life cycle. A critical post-transcriptional stage of the retroviral life cycle involves an interaction between the TAT protein and the TAR RNA sequence. TAR RNA is a RNA structure with a 3 base pair U-rich bulge. TAT protein contains an arginine rich RNA binding motif. The TAT protein binds with the TAR RNA region. The TAT protein promotes full length retroviral RNA transcript synthesis. Disruption of the TAT protein binding with the TAR RNA region inhibits full length retroviral RNA transcript synthesis. In some embodiments, the present invention provides methods and compositions for inhibiting TAT protein binding with TAR RNA region. In other embodiments, the present invention provides methods and compositions for inhibiting full length retroviral RNA transcript synthesis. In addition, the TAT protein facilitates RNA elongation by facilitating the engagement of RNA polymerase with retroviral RNA. Inhibition of the TAT protein results in disengagement of RNA polymerase from the retroviral RNA. In other embodiments, the present invention provides methods and compositions for inhibiting the engagement of RNA polymerase with retroviral RNA.

II. Exemplary Compounds

Exemplary Compounds of the Present Invention are Provided below.

In some embodiments, the compounds of the present invention have the structure: 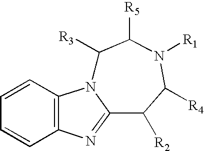
or its enantiomer, or a pharmaceutically acceptable salt, prodrug or derivative thereof; wherein R₁ functions through inhibiting viral DNA Polymerase, and comprises a linear or branched, saturated or unsaturated aliphatic chain having at least 2 carbons; wherein, R₂ functions through binding the viral RT palm subdomain of the p66 subunit, and comprises a hydroxy-methyl-ketone, a orthophosphate-methyl-ketone, a diphosphate-methyl-ketone, or a triphosphate-methyl-ketone; and wherein R₃, R4, and R₅ comprise a hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup.

The term “alkane” represents hydrocarbons, containing only single Carbon bonds, including cycloalkanes, and not being limited in length. The term “alkene” represents hydrocarbons, containing at least one double Carbon to Carbon bond, including cycloalkenes, and not being limited in length. The term “alkyne” represents hydrocarbons, containing at least one triple Carbon to Carbon bond, and not being limited in length. Further, it should be understood that the numerical ranges given throughout this disclosure should be construed as a flexible range that contemplates any possible subrange within that range. For example, the description of a group having the range of 1-10 carbons would also contemplate a group possessing a subrange of, for example, 1-3, 1-5, 1-8, or 2-3, 2-5, 2-8, 3-4, 3-5, 3-7, 3-9, 3-10, etc., carbons. Thus, the range 1-10 should be understood to represent the outer boundaries of the range within which many possible subranges are clearly contemplated. Additional examples contemplating ranges in other contexts can be found throughout this disclosure wherein such ranges include analogous subranges within.

The term “a moiety that participates in hydrogen bonding” as used herein represents a group that can accept or donate a proton to form a hydrogen bond thereby.

Some specific non-limiting examples of moieties that participate in hydrogen bonding include a fluoro, oxygen-containing and nitrogen-containing groups that are well-known in the art. Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached.

Thus, for example, the term “lower carbonyl” refers to inter alia, formaldehyde, acetaldehyde.

Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups.

It is also possible that the hydrogen-bond acceptor in the present invention can be the Π electrons of an aromatic ring.

The term “heterocyclic” represents, for example, a 3-6 membered aromatic or nonaromatic ring containing one or more heteroatoms. The heteroatoms can be the same or different from each other. Preferably, at least one of the heteroatoms is nitrogen. Other heteroatoms that can be present on the heterocyclic ring include oxygen and sulfur.

Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine.

Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine.

Examples of heterocyclic rings containing two different heteroatoms include, but are not limited to, phenothiazine, morpholine, parathiazine, oxazine, oxazole, thiazine, and thiazole.

Some specific examples of the benzimidazole compounds of this invention include:

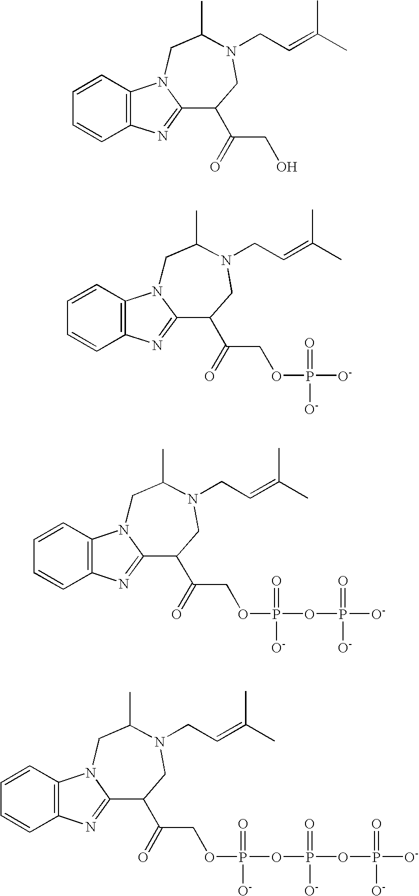

The stereochemistry of all derivatives embodied in the present invention is R, S, or racemic.

In summary, a large number of benzimidazole compounds and related compounds are presented herein. Any one or more of these compounds can be used alone, or in combination, to treat a variety of retroviral disorders as described elsewhere herein. The above-described compounds can also be used in drug screening assays and other diagnostic or research methods.

III. Pharmaceutical Compositions, Formulations, and Exemplary Administration Routes and Dosing Considerations

Exemplary embodiments of various contemplated medicaments and pharmaceutical compositions are provided below.

A. Preparing Medicaments

The compounds of the present invention are useful in the preparation of medicaments to treat a variety of conditions associated with retroviral infection.

In addition, the compounds are also useful for preparing medicaments for treating other disorders wherein the effectiveness of the compounds are known or predicted. Such disorders include, but are not limited to, immune system disorders (e.g., T-Cell Lymphoma, AIDS). Known methods may be used and techniques for preparing medicaments of the compounds of the present invention. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent (e.g., benzimidazole derivative), as defined above, together with a solid support or alternatively, together with one or more pharmaceutically acceptable carriers and optionally other therapeutic agents (e.g., antiviral agents). Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In preferred embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose)surface-active or dispersing agent. Molded tablets are made by molding in a suitable machine a mixture of the powdered compound (e.g., active ingredient) moistened with an inert liquid diluent. Tablets may optionally be coated or scored and maybe formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternative embodiments, topical formulations comprise patches or dressings such as a bandage or adhesive plasters impregnated with active ingredient(s), and optionally one or more excipients or diluents. In preferred embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include TWEEN 60, SPAN 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

C. Exemplary Administration Routes and Dosing Considerations

Various delivery systems are known and can be used to administer a therapeutic agents (e.g., benzimidazole derivatives) of the present invention, e.g., encapsulation in liposomes, microparticles, microcapsules, receptor-mediated endocytosis, and the like. Methods of delivery include, but are not limited to, intra-arterial, intramuscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals susceptible to or at risk of developing a retroviral disease (e.g., AIDS) and condition correlated with this. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is HIV-1, the additional agent can be a protease inhibitor. When the condition being treated is an autoimmune disorder, the additional agent can be an immunosuppressant or an anti-inflammatory agent. The additional agents to be co-administered, such as immunosuppressant, anti-inflammatory, and can be any of the well-known agents in the art, including, but not limited to, those that are currently in clinical use. The determination of appropriate type and dosage of radiation treatment is also within the skill in the art or can be determined with relative ease.

IV. Drug Screens

In some embodiments of the present invention, the compounds of the present invention, and other potentially useful compounds, are screened for an ability to inhibit RT activity. In other embodiments, the compounds of the present invention, and other potentially useful compounds, are screened for an ability to inhibit retroviral RNA synthesis. In preferred embodiments, the compounds of the present invention, and other potentially useful compounds, are screened for an ability to simultaneously inhibit both RT activity and RNA synthesis. A number of suitable screens for measuring RT activity and RNA synthesis are known in the art. In some embodiments, RT activity and RNA synthesis screens are conducted in in vitro systems. In other embodiments, these screens are conducted in in vivo or ex vivo systems.

In some embodiments, compounds are screened in retroviral cell culture or in vivo (e.g., non-human or human mammals) for their ability to modulate RT activity and RNA synthesis. Any suitable assay may be utilized, including, but not limited to, cell proliferation assays (Commercially available from, e.g., Promega, Madison, Wis. and Stratagene, La Jolla, Calif.) and cell based dimerization assays. (See e.g., Fuh et al., Science, 256:1677 [1992]; Colosi et al., J. Biol. Chem., 268:12617 [1993]).

The present invention also provides methods of modifying and derivatizing the compositions of the present invention to increase desirable properties (e.g., RT inhibition, and the like), or to minimize undesirable properties (e.g., nonspecific reactivity, toxicity, and the like). The principles of chemical derivatization are well understood. In some embodiments, iterative design and chemical synthesis approaches are used to produce a library of derivatized child compounds from a parent compound. In other embodiments, rational design methods are used to predict and model in silico ligand-receptor interactions prior to confirming results by routine experimentation.

V. Therapeutic Application

In particularly preferred embodiments, the compositions (e.g., benzimidazole derivatives) of the present invention provide therapeutic benefits to patients suffering from any one or more of a number of conditions (e.g., diseases characterized by retroviral infection, AIDS, T-Cell Lymphoma, etc.) by modulating (e.g., inhibiting or promoting) the activity of RT and RNA synthesis in affected cells or tissues.

In particularly preferred embodiments, the compositions of the present invention inhibit the activity of RT by either separately or simultaneously inhibiting RT, inhibiting RT polymerase, or inhibiting RNA synthesis. While the present invention is not limited to any particular mechanism, nor to any understanding of the action of the agents being administered, in some embodiments, the compositions of the present invention bind in the RT palm subdomain of the p66 subunit. In still other preferred embodiments, while the present invention is not limited to any particular mechanism, nor to any understanding of the action of the agents being administered, it is contemplated that the compositions of the present invention inhibit retroviral TAT protein from binding TAR RNA.

Accordingly, preferred methods embodied in the present invention, provide therapeutic benefits to patients by providing compounds of the present invention that modulate (e.g., inhibit or promote) the retroviral life cycle.

Thus, in one broad sense, preferred embodiments of the present invention are directed to the discovery that many diseases characterized by retroviral infection (e.g., AIDS) can be treated by modulating the activity of RT and DNA synthesis. The present invention is not intended to be limited, however, to the practice of the compositions and methods explicitly described herein. Indeed, those skilled in the art will appreciate that a number of additional compounds not specifically recited herein (e.g., non-benzimidazole derivatives) are suitable for use in the methods disclosed herein of modulating the activity of the retroviral life cycle.

The present invention thus specifically contemplates that any number of suitable compounds or chemical moieties presently known in the art, or developed later, can optionally find use in the methods of the present invention in combination with the present compounds or by incorporation into the present compounds. The present invention is not intended, however, to be limited to the methods or compounds specified above. In one embodiment, compounds potentially useful in the methods of the present invention may be selected from those suitable as described in the scientific literature. (See e.g., K. B. Wallace and A. A. Starkov, Annu. Rev. Pharmacol. Toxicol., 40:353-388 [2000]; A. R. Solomon et al., Proc. Nat. Acad. Sci. U.S.A., 97(26):14766-14771 [2000]).

In one aspect, derivatives (e.g., pharmaceutically acceptable salts, analogs, stereoisomers, and the like) of the exemplary compounds or other suitable compounds are also contemplated as being useful in the methods of the present invention.

Those skilled in the art of preparing pharmaceutical compounds and formulations will appreciate that when selecting optional compounds for use in the methods disclosed herein, that suitability considerations include, but are not limited to, the toxicity, safety, efficacy, availability, and cost of the particular compounds.

### EXAMPLES

### Example 1

Synthesis of Antiviral Agent

Preparation of 1 was accomplished through the following procedure. Preparation of 2 proceeded with a 5-step sequence starting from L-alanine. The coupling between fluoronitroaniline 3 and BOC mono-protected 1, 2- diaminopropropane 2 went smoothly under the typical nucleophilic aromatic substitution conditions (K₂CO₃ and DMF) to give the desired adduct 4. Subsequent reduction of 4 via hydrogenation cleanly yielded the desired diamine 5, which was then condensed with the aldehyde 6 in the presence of DDQ to afford the desired bezimidazole 7 in 59% yield over the three steps from 3 and 2. Deprotection of the TBDPS group was accomplished with TBAF in THF to give the desired alcohol 8 in 87% yield, which was subsequently subjected to a three-step cyclization sequence (activation of the alcohol 8 as the mesylate 9, deprotection of the BOC group, and cyclization) to provide the tricyclic core 10 in 91% yield over 3 steps from 8. The installation of the prenyl side chain was complete under the condition of NaH (2 equiv.) and prenyl bromide 11 (1.5 equiv.) to afford the core structure 12 in 76% yield, which was then acylated with methyl chlorooxylate 13 to afford the desired acylated benzimidazole 14 in 47% yield. The diol 15 was obtained by NaBH4 via a one-step reduction protocol in 69% overall yield from 14. Selective protection of the primary hydroxyl group as the TBS either 16 and subsequent oxidation of the remaining secondary alcohol under Swem conditions furnished the desired ketone 17. Finally, the synthesis of 1 was completed by deprotection of the TBS ether under the condition of aqueous acetic acid in THF.

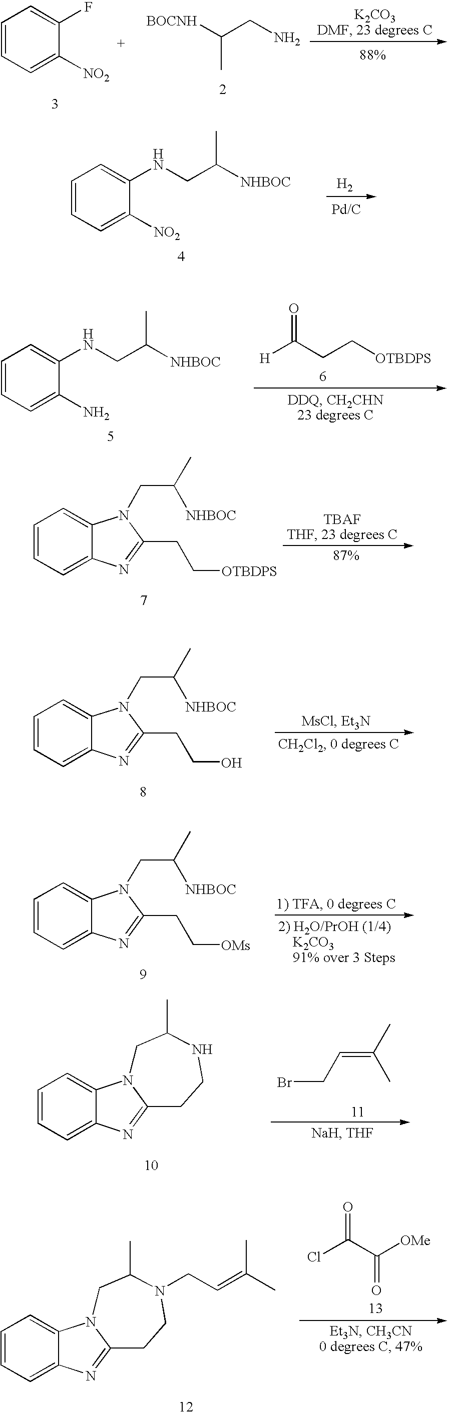

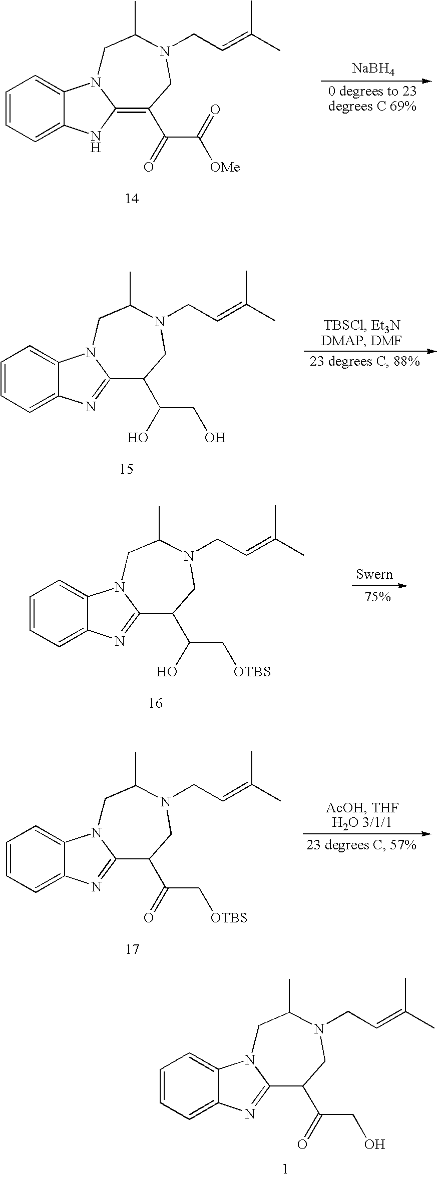

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A composition comprising the following formula: 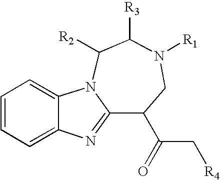
including its enantiomer and racemic mixtures; wherein R1 comprises a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxyl group, an orthophosphate group, a diphospate group, or a triphosphate group.

2. A composition comprising the following formula: 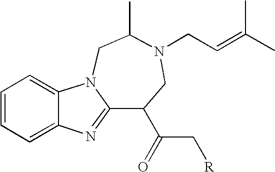
including its enantiomers and racemic mixtures; wherein R is a hydroxyl group, an orthophosphate group, a diphospate group, or a triphosphate group.

3. A composition comprising the following formula: 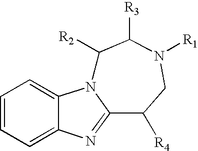
including its enantiomers and racemic mixtures; wherein R1 is a branched alkane, a branched alkene, or a branched alkyne; wherein R2 and R3 are selected from the group consisting of: hydrogen; CH₃; a linear or branched, saturated or unsaturated aliphatic chain having at least 2 carbons; a linear or branched, saturated or unsaturated aliphatic chain having at least 2 carbons, and having at least one hydroxy subgroup; a linear or branched, saturated or unsaturated aliphatic chain having at least 2 carbons, and having at least one thiol subgroup; a linear or branched, saturated or unsaturated aliphatic chain having at least 2 carbons, wherein said aliphatic chain terminates with an aldehyde subgroup; a linear or branched, saturated or unsaturated aliphatic chain having at least 2 carbons, and having at least one ketone subgroup; a linear or branched, saturated or unsaturated aliphatic chain having at least 2 carbons; wherein said aliphatic chain terminates with a carboxylic acid subgroup; a linear or branched, saturated or unsaturated aliphatic chain having at least 2 carbons, and having at least one amide subgroup; a linear or branched, saturated or unsaturated aliphatic chain having at least 2 carbons, and having at least one nitrile subgroup; a linear or branched, saturated or unsaturated aliphatic chain having at least 2 carbons, and having at least one amine subgroup; a linear or branched, saturated or unsaturated aliphatic chain having at least 2 carbons, and having at least one ether subgroup; a linear or branched, saturated or unsaturated aliphatic chain having at least 2 carbons, and having at least one halogen subgroup; or a linear or branched, saturated or unsaturated aliphatic chain having at least 2 carbons, and having at least one nitro subgroup; wherein R4 is a hydroxy-methyl-ketone, an orthophosphate-methyl-ketone, a diphosphate-methyl-ketone, or a triphosphate-methyl-ketone.
